(19) United States
(12) Patent Application Publication
(10) Pub. No.: US 2014/0135583 A1
(43) Pub. Date: May 15, 2014

(54) Intubation Tube

(71) Applicant: Manuel V. Moreno, Williamsport, PA (US)

(72) Inventor: Manuel V. Moreno, Williamsport, PA (US)

(21) Appl. No.: 13/676,733

(22) Filed: Nov. 14, 2012

(51) Int. Cl. A61M 16/04; A61B 1/267; A61B 1/06

(52) U.S. Cl. CPC A61M 16/0488; A61B 1/06; A61B 1/267
USPC 600/199

(57) ABSTRACT

Intubation device consisting of a flexible tube with a distal rubber-like pouch, wherein the pouch cavity communicates with the tube on one end. The other end of the tube is connected to a pressure sensor with light indicator. When it is squeezed it will compress the volume of the chamber and it displaces the air in the tube to trigger the pressure sensor in the proximal end to turn on the light indicator. When the external pressure on the pouch is released, the light indicator is off. The intubation guide has a handle and a curved blade. The tracheal tube follows the curve of the blade held by a tab that is pulled out when the user wants to release the tube. The tip of the introducer is aligned with the tip of the blade and has a light beam directed to the tip of the blade indicating blade location.

### CROSS REFERENCE TO RELATED APPLICATIONS

This application claims priority from U.S. Patent Application Ser. No. 61/559,440, entitled “Intubation Tube With Location Detection and Indicator”, filed on 14 Nov. 2011. The benefit under 35 USC §119(e) of the United States provisional application is hereby claimed, and the aforementioned application is hereby incorporated herein by reference.

### TECHNICAL FIELD OF THE INVENTION

The present invention relates generally to intubation tubes. More specifically, the present invention relates to intubation tubes and guides with means for determining location and indications of location.

### BACKGROUND OF THE INVENTION

Over 17 million tracheal intubations are performed annually in this country alone. Worldwide it is over 50 million. While it is a routine procedure, intubations are not without difficulty and up to 10 percent result in complications, some are very serious. Management of difficult airway remains the most relevant and challenging task for anesthesia care providers. A common factor preventing successful intubation is the inability to visualize the vocal cords during the performance of direct laryngoscopy, the most commonly utilized technique.

The use of fiber optic devices and tracheal aids has improved the outcome in the majority of cases. However, there are certain anatomies, where visualization of the vocal cords is extremely difficult or impossible even among experienced practitioners. Many times it involves neck manipulation where it is limited and sometimes harmful in cases of spine pathology or trauma. Forced laryngoscopy can cause harm to delicate cardiac patients.

Management of difficult airway remains the most relevant and challenging task for anesthesia care providers. A common factor preventing successful intubation is the inability to visualize the vocal cords during the performance of Direct Laryngoscopy, the most commonly utilized technique.

Almost all technology developed to achieve tracheal intubation are based on visualization of the glottis. The use of fiber optic devices to visualize the glottis has improved the outcome in the majority of cases when Direct Laryngoscopy fails. However, improved laryngeal view does not necessarily translate into increased intubation success. It is not easy to maneuver a tracheal tube in a 3 dimensional anatomy with a 2 dimensional view of the larynx using Video Laryngoscopy. It requires a lot of training Blood or secretions can impair the video laryngoscopy view.

However, many have successfully intubated the trachea without seeing it. Unfortunately, success with what we call “blind” intubation cannot be confirmed until the user connect the tracheal tube to a CO2 monitor & the user need to squeeze the breathing bag to confirm its presence. This could fill the stomach with air with a potentially serious negative consequence, like gastric aspiration, especially with repeated attempts.

Many times tracheal intubation involves neck manipulation where it is limited and sometimes harmful in cases of spine pathology or trauma. Forced laryngoscopy can cause harm to delicate cardiac patients.

Therefore, what is needed is a tracheal intubation guide with location detection and an external indicator. This reduces the number of complications and assists the user in locating and properly placing the intubation tube, using external landmarks, namely the laryngeal prominence and Cricoid, which can be seen or easily be palpable.

### SUMMARY OF THE INVENTION

Intubation device consisting of a flexible tube with a distal rubber-like pouch, wherein the pouch cavity communicates with the tube on one end. The other end of the tube is connected to a pressure sensor with external light indicator. This will form an air-tight chamber. The pouch has a good memory. When it is squeezed it will compress the volume of the chamber and it displaces the air in the tube to trigger the pressure sensor in the proximal end to turn on the external light indicator. When the external pressure on the pouch is released it will spring back to its original shape, the pressure in the air tight chamber will go back to the original pressure and the external light indicator will turn off.

The introducer is inserted in the tracheal tube with its terminal pouch just in front of the tube tip. The technique is to press the Cricoid to compress the esophagus so that if the pouch happens to be in it, the external light indicator will be on showing the operator that it is not in the trachea, where the user want it to be. Pressure on the Cricoid does not compress the trachea so when the pouch is in it the external light indicator will not be on.

The present invention also includes an intubation guide. The intubation guide of the present invention has a handle and a curved blade. It has a u-shaped groove at the back from the handle to the blade, which accommodates the tracheal tube with the introducer. The tracheal tube follows the curve of the blade held by a tab which can be pulled out easily when the user wants to release the tube. The tip of the introducer is aligned with the tip of the blade.

The handle of the intubation guide of the present invention has a light beam directed to the tip of the blade. When the blade is inside the pharyngeal cavity, the external light beam can be directed towards the Laryngeal Prominence (Adam's apple). This will give the user a good indicator where the tip of the blade is. Since the introducer tip with the pouch is aligned with the blade tip the user has an indicator telling the user that the pouch is in front of the laryngeal opening.

### BRIEF DESCRIPTION OF THE DRAWINGS

The accompanying drawings, which are incorporated herein a form a part of the specification, illustrate the present invention and, together with the description, further serve to explain the principles of the invention and to enable a person skilled in the pertinent art to make and use the invention.

FIG. 1 illustrates the current method for intubation and insertion of a tracheal tube as known in the prior art'

FIG. 2 illustrates the physical inducer apparatus of the present invention;

FIG. 3 is a side view of the inducer apparatus of the present invention;

FIG. 4 is an anatomical illustration;

FIG. 5 is an anatomical illustration of the device of the present invention being inserted;

FIGS. 6-10 are an anatomical illustrations of the method for inserting the device of the present invention; and

FIG. 11 is an anatomical illustration of the device properly inserted.

### DETAILED DESCRIPTION OF THE INVENTION

In the following detailed description of the invention of exemplary embodiments of the invention, reference is made to the accompanying drawings (where like numbers represent like elements), which form a part hereof, and in which is shown by way of illustration specific exemplary embodiments in while the invention may be practiced. These embodiments are described in sufficient detail to enable those skilled in the art to practice the invention, but other embodiments may be utilized and logical, mechanical, electrical, and other changes may be made without departing from the scope of the present invention. The following detailed description is, therefore, not to be taken in a limiting sense, and the scope of the present invention is defined only by the appended claims.

In the following description, numerous specific details are set forth to provide a thorough understanding of the invention. However, it is understood that the invention may be practiced without these specific details. In other instances, well-known structures and techniques known to one of ordinary skill in the art have not been shown in detail in order not to obscure the invention. Referring to the figures, it is possible to see the various major elements constituting the apparatus of the present invention.

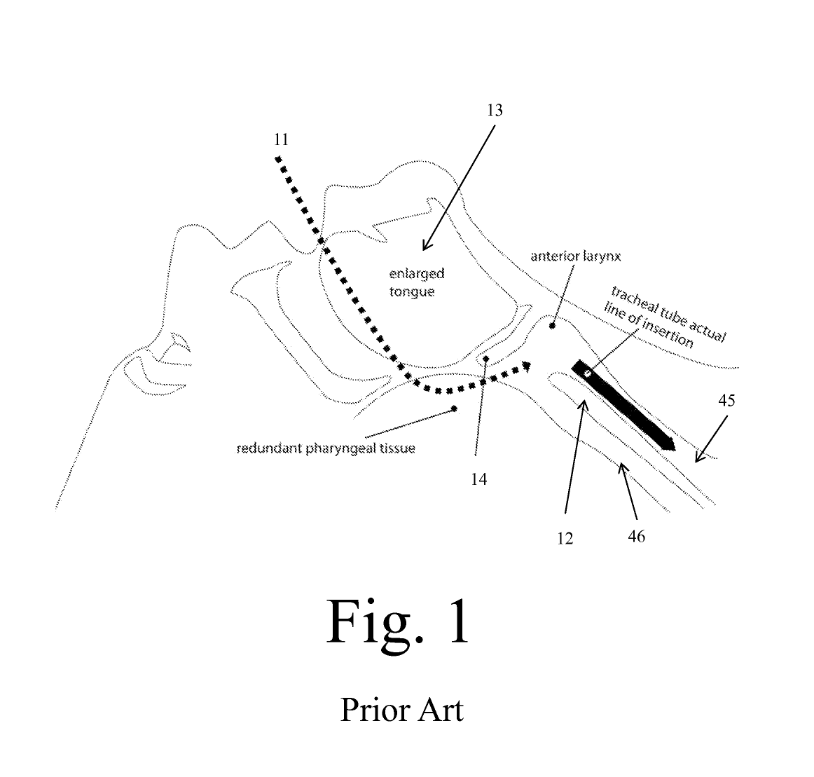

Now referring to the Figures, the embodiment of the intubation tube with location detection and an indicator is show. Many times tracheal intubation involves neck manipulation where it is limited and sometimes harmful in cases of spine pathology or trauma. Forced laryngoscopy can cause harm to delicate cardiac patients. FIG. 1 illustrates the anatomy of a difficult intubation and shows why extending the neck, in order to get a view of the glottis, would compound difficulty of inserting the Tracheal tube.

The path of the tracheal tube 11 is shown where the tracheal tube is inserted when extending the neck in a difficult anatomy. The tube 1 approaches the trachea tissue 12 at an angle, causing extreme force on the tongue 13. It is the extreme force on the tongue, in an attempt to visualize the glottis, that can distorts the anatomy.

The present invention is a form of blind intubation since the user doesn't need to see the glottis in order to successfully insert a tube into the trachea. The big advantage here is the user already has a confirmation that the user is in the trachea before the user connect the tube to a breathing bag.

Therefore, what is needed is a Tracheal Intubation Guide with location detection and an external indicator. This reduces the number of complications and assists the user in locating and properly placing the intubation tube, using external landmarks, namely the laryngeal prominence & Cricoid, which can be seen or easily be palpable.

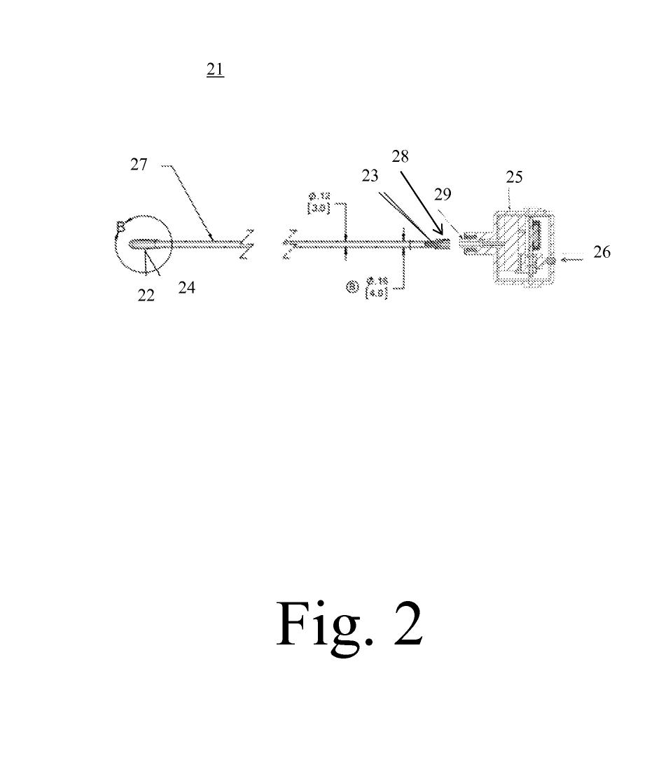

Now referring to FIG. 2, the Introducer 21 consists of a flexible tube 27 with a distal rubber-like pouch 22, wherein the pouch cavity 24 communicates with the tube 27 on one end. The other end of the tube 23 is connected to a pressure sensor 25 with external light indicator 26. This will form an air-tight chamber. The pouch 22 has a good memory. When it is squeezed it will compress the volume of the chamber and it displaces the air in the tube 27 to trigger the pressure sensor 25 in the proximal end to turn on the external light indicator 26. When the external pressure on the pouch 22 is released it will spring back to its original shape, the pressure in the air tight chamber will go back to the original pressure and the external light indicator 26 will turn off.

The Introducer 21 is inserted in tracheal tube with its terminal pouch 22 just in front of the tube tip. The technique is to press the Cricoid to compress the esophagus so that if the pouch 22 happens to be in it, the external light indicator 26 will be on showing the operator that it is not in the trachea, where the user want it to be. Pressure on the Cricoid does not compress the trachea so when the pouch 22 is in it the external light indicator 26 will not be on.

The external pouch 22 is made from a rubber like or similar material and deforms by external pressure. The pouch 22 returns to its original shape when external pressure is off.

The tube 27 is connected to the pressure sensor 25 by a Luer lock. The tube end 23 includes a female Luer lock 28 while the pressure sensor 25 is comprised of a male Leur lock 29, which provides an air tight connection between the tube 22 and pressure sensor 25 allowing for quick and easy replacement of either component.

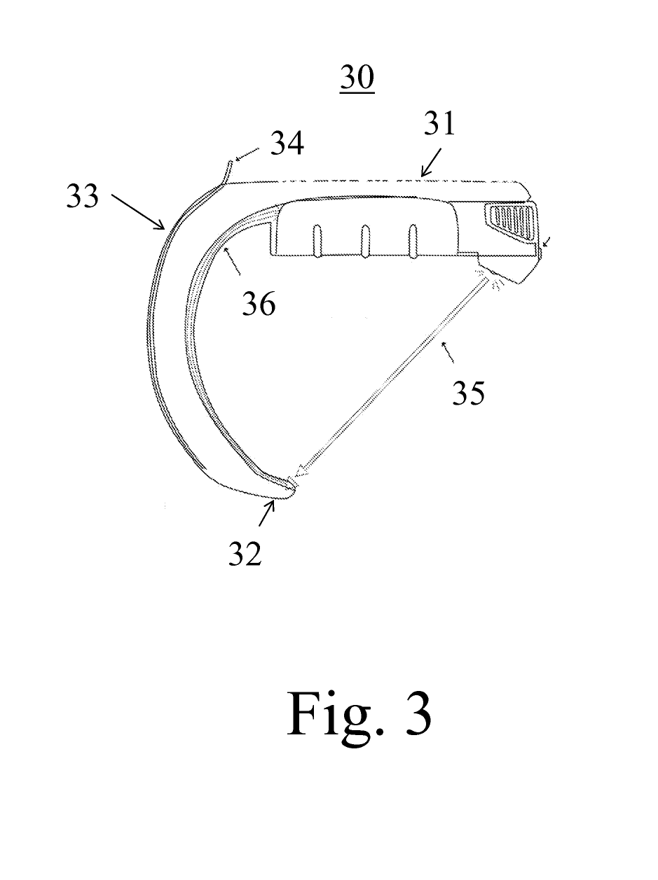

The present invention also includes an intubation guide 30 as shown in FIG. 3. The intubation guide 30 of the present invention has a handle 31 and a curved blade 32. It has a u-shaped groove 33 at the back from the handle 31 to the blade 32, which accommodates the tracheal tube 27 with the introducer 21 retained within the tracheal tube 27. The tracheal tube 27 follows the curve of the blade 32 held by a tab 34 which can be pulled out easily when the user wants to release the tube 27. It is not the introducer 27 that is desired to be released during use of the device, it is the tracheal tube 27 that fits in the u-shaped groove 33 of the intubation guide 30. The introducer 27 is in the tracheal tube 27 loosely. The tip of the introducer 21 is aligned with the tip of the blade 32.

The handle 31 of the intubation guide 30 of the present invention has a light beam 35 directed to the tip of the blade 32. When the blade 32 is inside the pharyngeal cavity, the external light beam 35 can be directed towards the Laryngeal Prominence (Adam's apple). This will give the user a good indicator where the tip of the blade 32 is. Since the introducer tip with the pouch 22 is aligned with the blade tip 32 the user has an indicator telling the user that the pouch 22 is in front of the laryngeal opening. The intubation guide 30 of the present invention also consists of a transparent plastic material 36 that acts as a fiber optic transporting light into the oral and pharyngeal cavity.

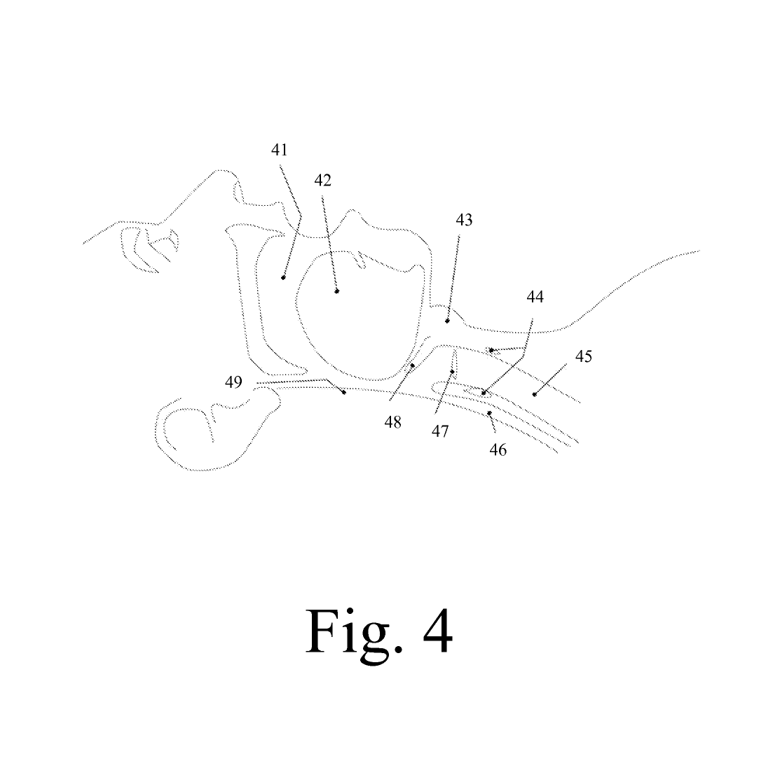

Now referring to FIG. 4, the basic anatomy of a person on while the device is to be used is illustrated. Physical landmarks are identified as the oral cavity 41, tongue 42, laryngeal prominence (Adam's apple) 43, cricoid cartilage 44, trachea 45, esophagus 46, vocal cords 47, epiglottis 48, and posterior pharyngeal wall 49.

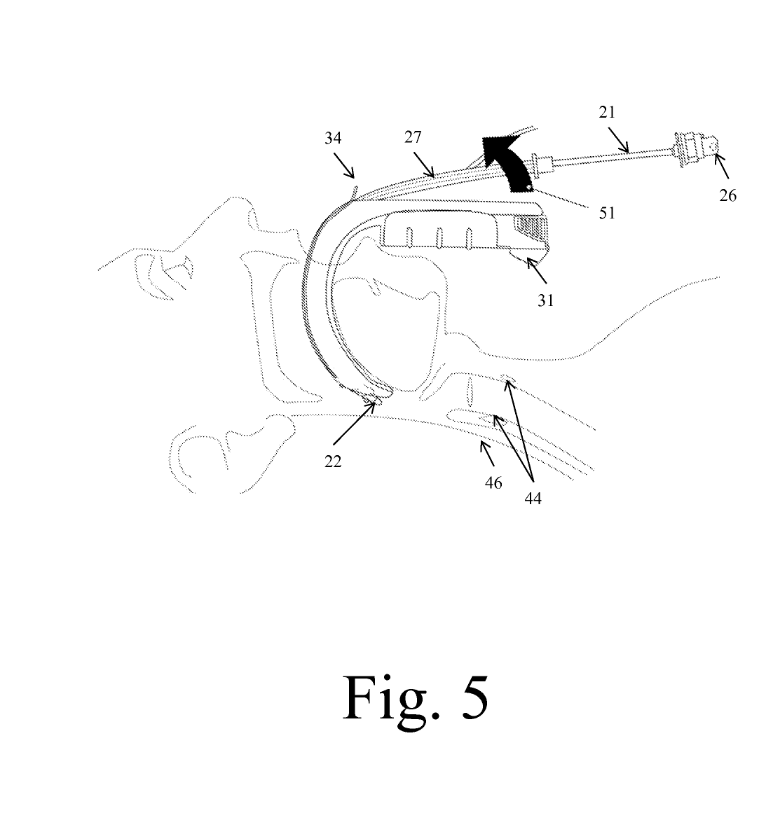

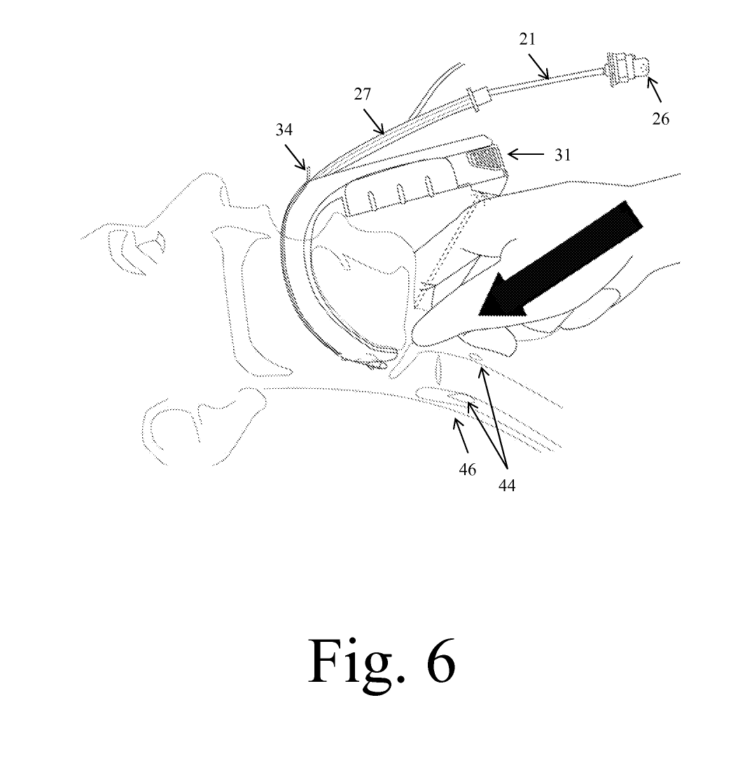

Now referring to FIGS. 5-10, the method of using the device of the present invention is illustrated. First, the blade is inserted into the oral cavity 41 of the patient. The handle 31 is moved in the direction of the arrow 51 to prepare for the next move. External landmarks are determined by using the thumb and index finger to grasp the laryngeal prominence to align it with the beam of light that provides a guide to the location of the bald tip in the pharynx 61. The fixed middle finder presses on the cricoid 44 to compress the esophagus 46. The direction of force is important to minimize tracheal compression that could potentially light up the external light indicator 26.

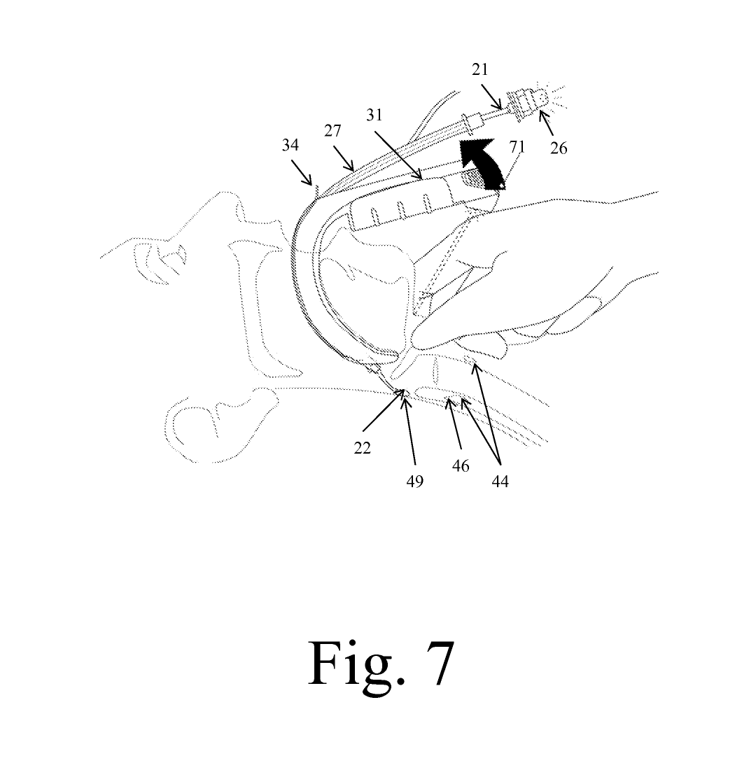

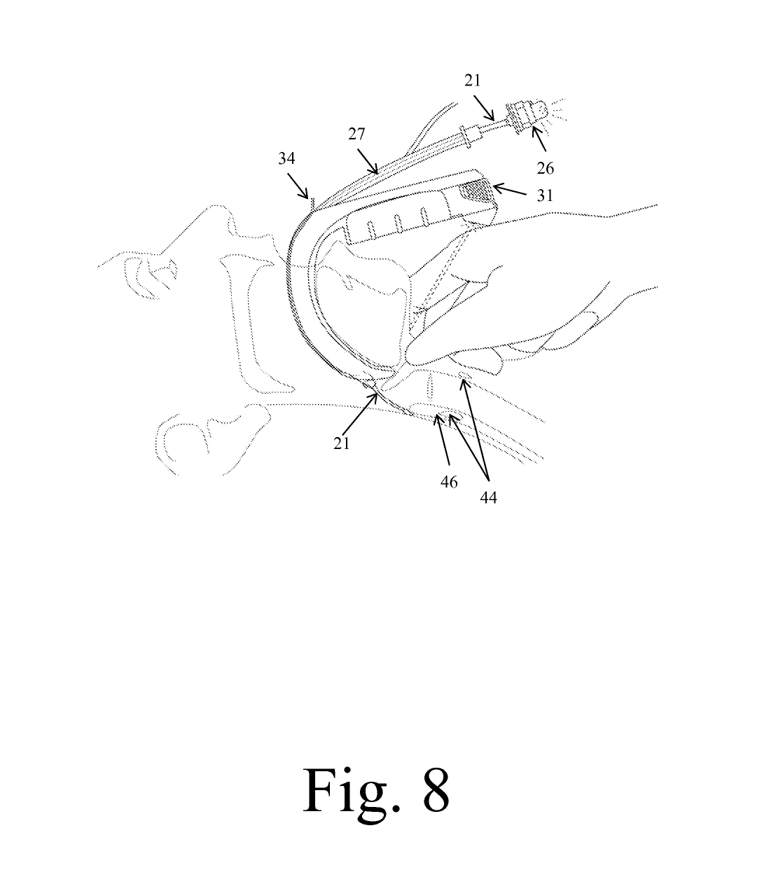

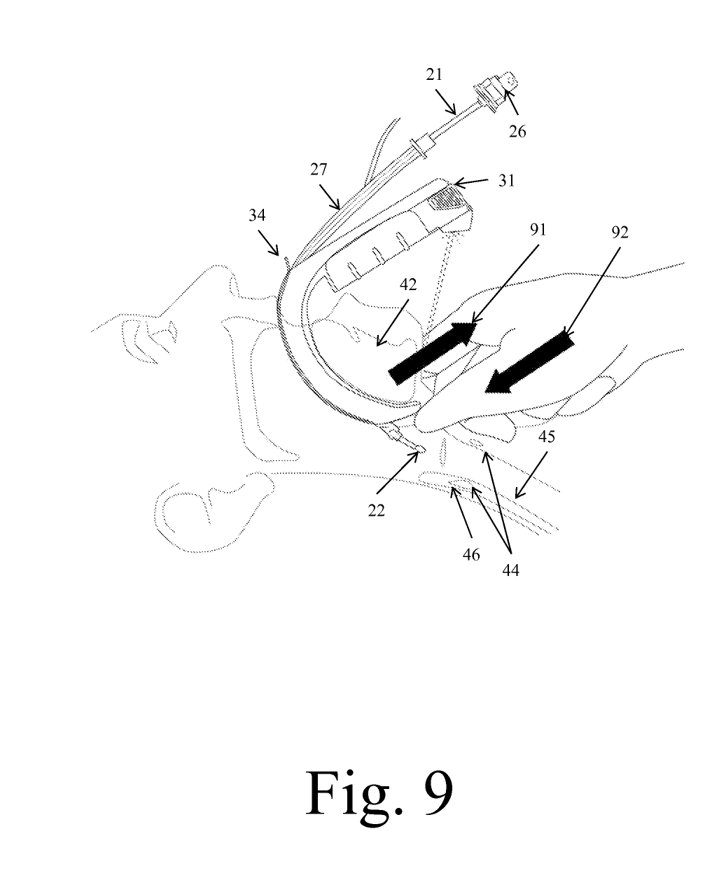

Now referring to FIG. 7, in a first trial the soft end or pouch 22 of the introducer 21 hits the posterior pharyngeal wall 49 causing the external light indicator 26 to turn on or light up. The introducer 21 is then pulled back slightly one or two centimeters or until the external light indicator 26 is off. As shown by the arrow 71, the handle is moved slightly in the direction of the arrow 71 to prepare for the next move. In a second trial, shown in FIG. 8, the introducer 21 hits the compressed esophagus and the external light indicator 26 turns on or lights up. The introducer 21 is then withdrawn slightly until the external light indicator 26 to turns off and the user is now ready for the third move. In FIG. 9, as the introducer 21 is withdrawn slightly and the external light indicator 26 to turns off, the handle is moved in the direction completely opposite to the cricoid pressure to compress the base of the tongue as shown by the arrows 91 and 92. This maneuver lifts the epiglottis so the introducer enters the trachea 45 unimpeded.

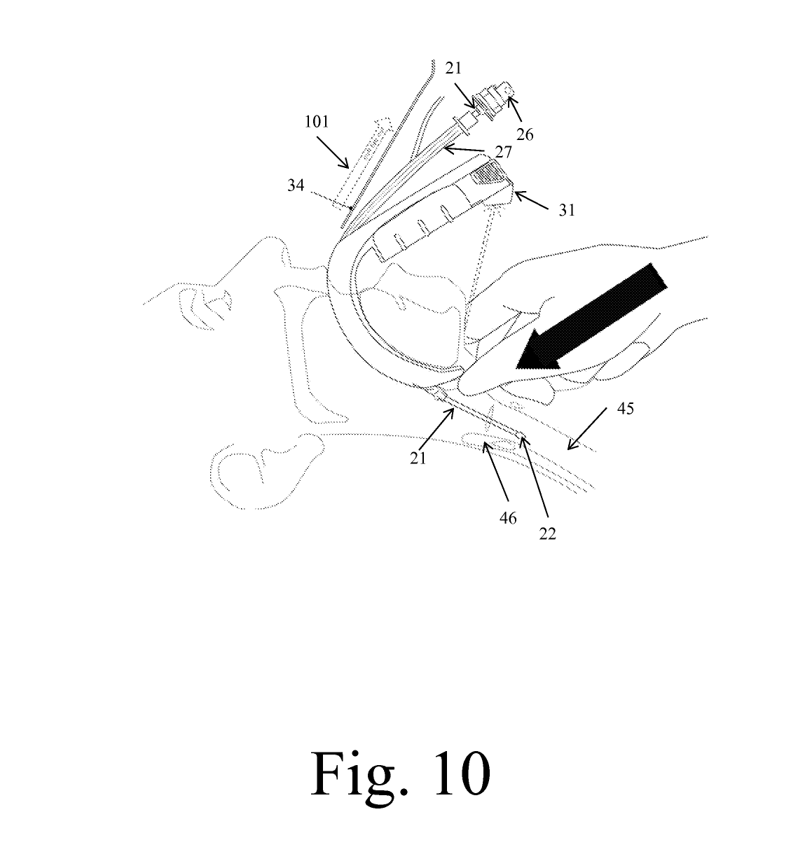

As shown in FIG. 10, the introducer 21 tip with the pouch 22 finds the trachea 45, the only location where the soft tip and its pouch 22 is not deformed. This is confirmed by the fact that the external light indicator 26 does not light up. The pull tab 34 is withdrawn as shown by arrow 101 to free the tracheal tube 27 from the handle 31.

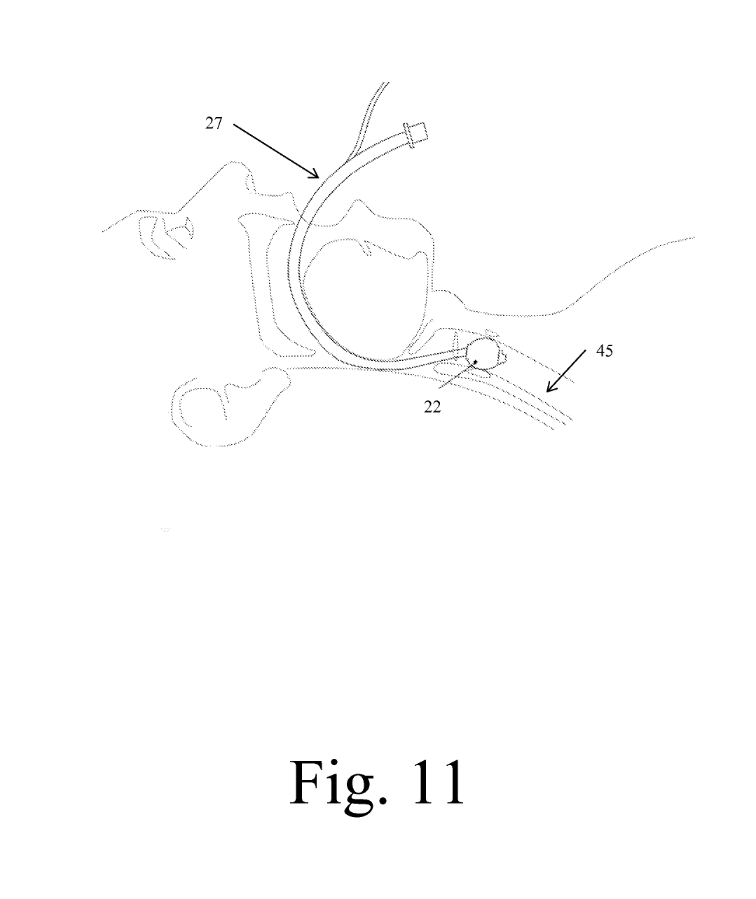

Finally, in FIG. 11, the tracheal tube 27 is shown in place in the trachea 45. The pouch 22 is inflated and the introducer 21 and handle 31 are removed.

Thus, it is appreciated that the optimum dimensional relationships for the parts of the invention, to include variation in size, materials, shape, form, function, and manner of operation, assembly and use, are deemed readily apparent and obvious to one of ordinary skill in the art, and all equivalent relationships to those illustrated in the drawings and described in the above description are intended to be encompassed by the present invention.

Furthermore, other areas of art may benefit from this method and adjustments to the design are anticipated. Thus, the scope of the invention should be determined by the appended claims and their legal equivalents, rather than by the examples given.

The embodiments of the invention in which an exclusive property or privilege is claimed are defined as follows:

1. An intubation tube guide device comprising:
an introducer comprised of:
a flexible tube;
a rubber-like pouch, wherein the pouch cavity communicates with the flexible tube on one end;
the opposing end of the tube is connected to a pressure sensor with external light indicator;
the pouch, and pressure sensor form an air-tight chamber within the tube; and
an intubation guide comprised of:
a handle;
a curved blade;
a u-shaped groove located at the outside of the curved blade from the handle, which accommodates the tube with the introducer;
a pull tab attached to the blade to keep the tracheal tube in place following the curve of the blade;
a transparent plastic material located on the inside curved blade that acts as a fiber optic transporting light into the oral and pharyngeal cavity;
the tube follows the curve of the blade held by a tab which can be pulled out easily when the user wants to release the tube;
the tip of the introducer is aligned with the tip of the blade;
the handle of the intubation guide of the present invention has a light beam directed to the tip of the blade; and
when the blade is inside the pharyngeal cavity, the external light beam can be directed towards the Laryngeal Prominence (Adam's apple).

2. The device of claim 1, wherein
when the pouch is squeezed it will compress the volume of the chamber and it displaces the air in the tube to trigger the pressure sensor in the opposing end to turn on the external light indicator; and
when the external pressure on the pouch 22 is released it will spring back to its original shape, the pressure in the air tight chamber will go back to the original pressure and the external light indicator will turn off.

3. The device of claim 1, wherein the flexible pouch is made from a material that provides a memory to its original shape.

4. The device of claim 3, wherein
the pouch is made from a rubber like or similar material and deforms by external pressure; and
the pouch returns to its original shape when external pressure is off

5. The device of claim 3, wherein
the tube is connected to the pressure sensor by a Luer lock which provides an air tight connection between the tube and pressure sensor;
the tube end includes a female Luer lock; and
the pressure sensor is comprised of a male Leur lock.

6. The device of claim 1, wherein the tab is pulled out to release the tracheal tube from the blade so it can be pushed easily into the trachea.

7. A method for using an intubation device comprising the steps of:
providing an intubation tube guide device comprising:
an introducer comprised of:
a flexible tube;
a rubber-like pouch, wherein the pouch cavity communicates with the flexible tube on one end;
the opposing end of the tube is connected to a pressure sensor with external light indicator;
the tub, pouch, and pressure sensor form an air-tight chamber; and
a intubation guide comprised of:
a handle;
a curved blade;
a u-shaped groove located at the outside of the curved blade from the handle, which accommodates the tube with the introducer;
a transparent plastic material located on the inside curved blade that acts as a fiber optic transporting light into the oral and pharyngeal cavity;
the tube follows the curve of the blade held by a tab which can be pulled out easily when the user wants to release the tube;
the tip of the introducer is aligned with the tip of the blade;
the handle of the intubation guide of the present invention has a light beam directed to the tip of the blade; and
when the blade is inside the pharyngeal cavity, the external light beam can be directed towards the Laryngeal Prominence (Adam's apple);
inserting the tube into the oral cavity of a patient;
moving the handle in an upward motion to prepare for the next move;
determining external landmarks are determined by using the thumb and index finger to grasp the laryngeal prominence;
aligning the laryngeal prominence with the beam of light that provides a guide to the location of the blade tip in the pharynx;
pressing on the cricoid to compress the esophagus;
placing the soft end or pouch of the introducer against the posterior pharyngeal wall causing the external light indicator to turn on or light up;
pulling the introducer back slightly one or two centimeters or until the external light indicator is off;
handle in an upward motion to prepare for the next move;
inserting the introducer further until it hits the compressed esophagus and the external light indicator turns on or lights up;
withdrawing the introducer until the external light indicator to turns off;
moving the handle \ in the direction completely opposite to the cricoid pressure to compress the base of the tongue; and
lifting the epiglottis so the introducer enters the trachea unimpeded.

8. The method of claim 7, further comprising the steps of:
confirming the location of the introducer tip and pouch in the trachea by the fact that the external light indicator does not light up; and
withdrawing the pull tab to free the tube from the handle.

9. The method of claim 8, further comprising the steps of:
inflating the pouch; and
removing the introducer and handle.
